(19) United States
(12) Patent Application Publication
(10) Pub. No.: US 2025/0221490 A1
(43) Pub. Date: Jul. 10, 2025

(54) ORTHOTIC KIT AND AN ORTHOTIC FORMED THEREFROM

(71) Applicant: Rhys BROWN, Moe Victoria (AU)

(72) Inventor: Rhys BROWN, Moe Victoria (AU)

(21) Appl. No.: 18/855,644

(22) PCT Filed: Mar. 30, 2023
(86) PCT No.: PCT/AU2023/050239

§ 371 Date: Oct. 10, 2024

(30) Foreign Application Priority Data
Apr. 12, 2022 (AU) 2022900977

(51) Int. Cl. A43B 13/16; A43B 3/00; A43B 7/144; A61F 5/01

(52) U.S. Cl. CPC A43B 13/16; A43B 3/0047; A43B 7/144; A61F 5/0111

(57) ABSTRACT

A kit for forming a foot orthotic, the kit comprising: two or more mid-foot components, each having a support region that is arched to respective extents such that each mid-foot component provides respectively different levels of arch support; and one or more rear-foot components configured to provide heel support, wherein the or each rear-foot component is removably attachable to each of the mid-foot components to form respective orthotics providing differing levels of arch and/or heel support.

### FIELD OF THE INVENTION

Disclosed herein is a kit for forming a foot orthotic, along with foot orthotics formed from said kit.

### BACKGROUND

Foot orthotics are inserted into shoes and worn to treat and/or provide support to a wearer's feet. In so doing, foot orthotics may also ameliorate associated issues such as knee and back pain. Such orthotics can better distribute ground forces received by the foot and can help with correcting foot posture.

Foot orthotics are commonly custom made, which may involve taking a mould of a patient's feet. Such custom-made orthotics are not amenable for mass production and can be relatively labour and time-consuming to make, which translates into increased costs for the end user.

Mass produced shoe insoles represent a more cost-effective alternative to custom orthotics. However, because such insoles are not custom fit to a wearer's foot, they may not be suitable or ideal for a wide range of users.

There is a need to address the above, and/or at least provide a useful alternative.

### SUMMARY

According to a first aspect of the present invention, there is provided a kit for forming a foot orthotic, the kit comprising:
two or more mid-foot components, each having a support region that is arched to respective extents such that each mid-foot component provides respectively different levels of arch support; and
one or more rear-foot components configured to provide heel support,
wherein the or each rear-foot component is removably attachable to each of the mid-foot components to form respective orthotics providing differing levels of arch and/or heel support.

Examples of the kit may comprise two or more rear-foot components, each having a heel cup region that is angled to respective extents such that each rear-foot component provides respectively different levels of heel correction.

In example kits, each mid-foot component comprises a rearward region configured for removable attachment to the rear-foot component(s).

In example kits, an underside of the mid-foot components and an underside of the rear-foot component(s) comprise respective complementarily shaped features to facilitate removable attachment between the mid and rear-foot components to form the respective orthotic.

In example kits, the complementarily shaped features comprise teeth and corresponding grooves into which they are insertable.

In example kits, the mid-foot components and the rear-foot component(s) are formed from a relatively rigid plastic material.

In example kits, one or more of the mid-foot components comprise a relatively flat forward landing for receiving a front of the wearer's foot.

In example kits, each component comprises an upper cushioning layer for receiving the wearer's foot.

Examples of the kit may comprise:
a first mid-foot component, the support region of which is arched to a low extent so as to provide a low level of arch support;
a second mid-foot component, the support region of which is arched to a medium extent so as to provide a medium level of arch support; and
a third mid-foot component, the support region of which is arched to a high extent so as to provide a high level of arch support.

Examples of the kit may comprise:
a first rear-foot component, the heel cup region of which is substantially flat so as to provide substantially no heel correction;
a second rear-foot component, the heel cup region of which is angled to a first extent so as to provide a mild level heel correction; and
a third rear-foot component, the heel cup region of which is angled to a second extent which is larger than the first extent so as to provide a high level of heel correction.

According to a second aspect of the present invention, there is provided a foot orthotic formed from a kit according to a first aspect of the present invention.

### BRIEF DESCRIPTION OF THE DRAWINGS

An embodiment of the invention will now be described, by way of example only, with reference to the accompanying drawings in which:

FIG. 1 is a top perspective view of mid-foot components and rear-foot components of a foot orthotic kit in accordance with examples of the present subject matter;

FIG. 2 is a top perspective view of the components of FIG. 1 assembled together to form three respective foot orthotics;

FIGS. 3A and 3B are, respectively, front perspective views of respective full-length mid-foot components;

FIG. 4A is a front perspective view of an example three-quarter length mid-foot component;

FIG. 4B is a front perspective view of an example rear-foot component;

FIG. 4C is a top view of the rear-foot components of FIG. 1;

FIGS. 5A to 5C show front and front cross-sectional views of the respective rear-foot components of FIG. 1, the cross-sectional views taken along the line A-A of FIG. 4C;

FIG. 6A is a bottom perspective view of a mid-foot component and a rear-foot component prior to assembly;

FIG. 6B is a bottom perspective view of the components of FIG. 5A assembled together to form a foot orthotic;

FIG. 7 is a bottom view of a mid-foot component and a rear-foot component prior to assembly;

FIG. 8 is a bottom view of the components of FIG. 6 assembled together to form a foot orthotic;

FIG. 9A is a front perspective view of mid and rear-foot components prior to assembly according to embodiments of the present invention; and

FIG. 9B is a front perspective view of mid and rear-foot components prior to assembly according to other embodiments of the present invention.

### DETAILED DESCRIPTION

The subject matter of the present specification relates to a kit for forming one or more foot orthotics. The kit may comprise different mid-foot components for providing different levels of arch support, and different rear-foot components for providing different levels of heel correction. Beneficially, the different mid-foot components and the different rear-foot components can be removably attached to one another to form numerous orthotics which differ in the level of arch support and heel correctiveness they provide.

For example, a kit comprising three mid-foot components providing respective levels of arch support and three rear-foot components providing respective levels of heel correction can be used to generate nine unique orthotics. In order words, the present kit preferably provides a range of different mid-foot components and a range of different rear-foot components, the mid and rear-foot components being capable of removably securing to one another so as to form numerous different orthotics which provide a level of arch support and heel correction that is appropriate to the patient/wearer. As such, it is envisaged that the mid and rear-foot components of the kit may be mass produced and easily attached to one another to form relatively customised yet cost-efficient foot orthotics.

Of course, the kit may comprise many more and different mid-foot and rear-foot components, particularly to account for both the left and right feet, as well as across feet sizes. For example, a comprehensive orthotic kit might include a range of different mid and rear-foot components for each foot, and for each foot size. However, the high-level principle of mixing and matching mid-foot components with rear-foot components to form a custom orthotic remains the same. An example orthotic kit, along with example orthotics that can be formed therefrom, will now be described with reference to the Figures.

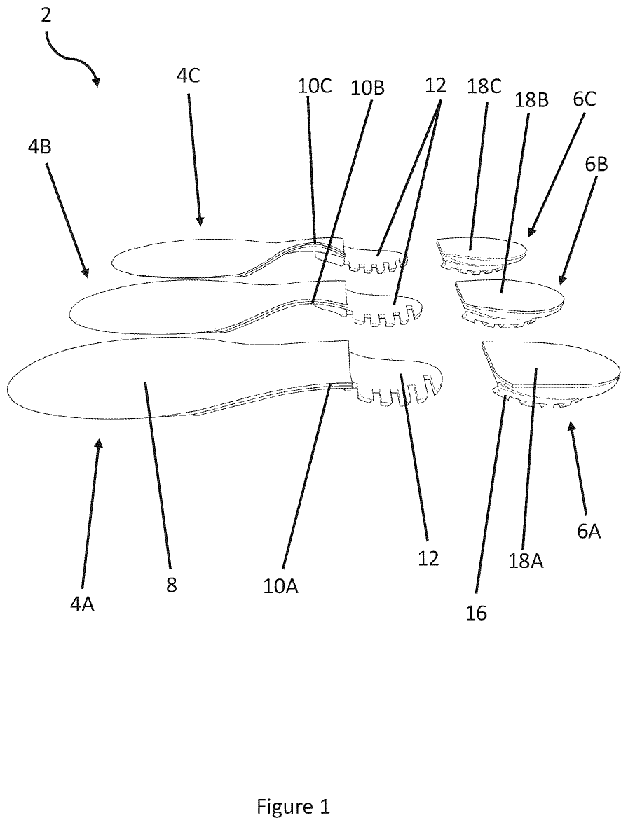

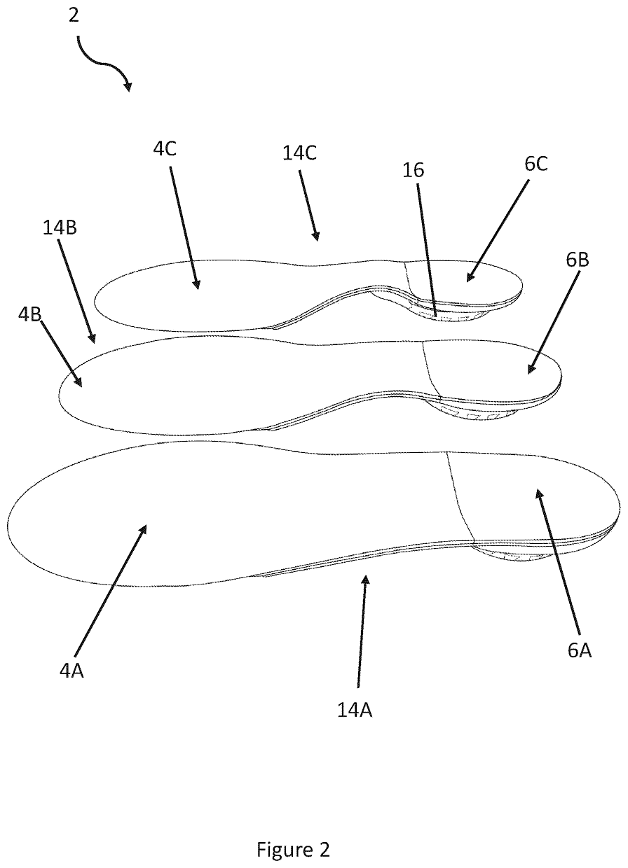

FIG. 1 shows an example foot orthotic kit 2 comprising three mid-foot components 4 and three rear-foot components 6. Each of the mid-foot components 4 have a relatively flat forward landing 8 for receiving a front of the wearer's foot, a middle support region 10 that is arched to a certain extent to provide arch support, and a rear attachment region 12 via which the rear-foot components 6 can be attached to form a complete orthotic 14, as shown in FIG. 2.

In the depicted example, the support region 10A of a first mid-foot component 4A is arched upwardly to a relatively low extent such that it provides a relatively low level of arch support. A second mid-foot component 4B has a support region 10B that has a more pronounced arch so as to provide an increased or moderate level of arch support. The support region 10C of a third mid-foot component 4C has an even more pronounced arch, such that it provides a high level of arch support.

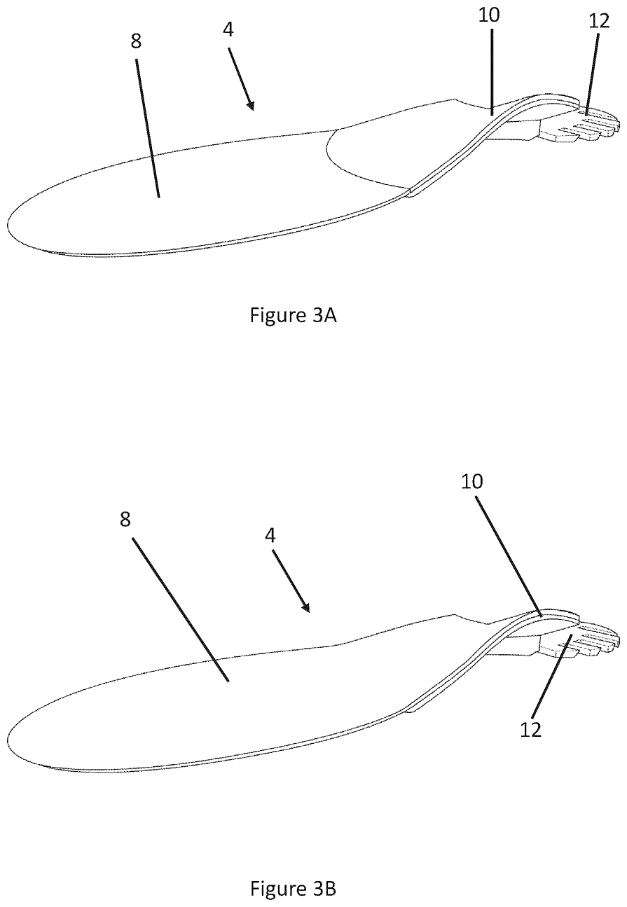

In FIGS. 1 and 2, the mid and rear-foot components 4, 6 comprise an upper cushioning layer for receiving the wearer's foot. However, the components 4, 6 need not comprise such a cushioning layer, particularly if the wearer prefers to supply their own. FIG. 3A shows an example of “full-length” mid-foot component 4, having a relatively flat front landing portion 8 which is formed from a soft material such as EVA foam. Meanwhile, the middle support region 10 and the rear attachment region 12 may be formed from a relatively rigid material, such as PA11 Nylon or Polypropylene, which is sufficiently strong such that at least the arched support region 10 of the mid-foot component 4 maintains its arched shape under the weight of the wearer so as to provide the desired arch support. FIG. 3B shows another embodiment of a “full-length” mid-foot component 4, wherein the entire component is integrally formed from the relatively rigid material.

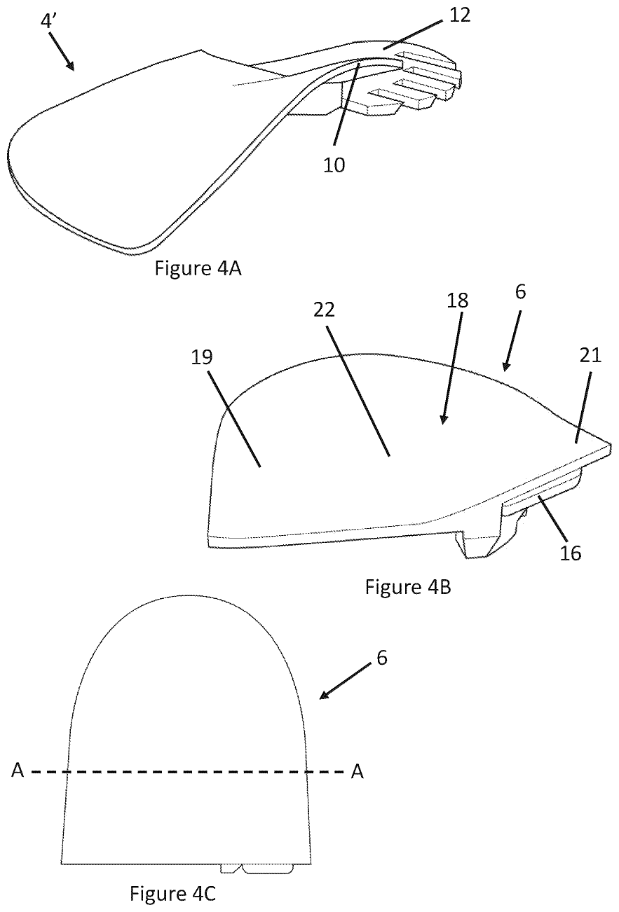

FIG. 4A shows another embodiment of a mid-foot component 4′. This may be referred to as a “¾” length component in that it does not include the relatively flat and frontal landing for receipt of the front of the wearer's foot. Instead, this embodiment of the mid-foot component 4′ is primarily formed from the arched support region 10 at a forward end thereof, and the rearward connection region 12 for attachment to a rear-foot component 6, as will be discussed.

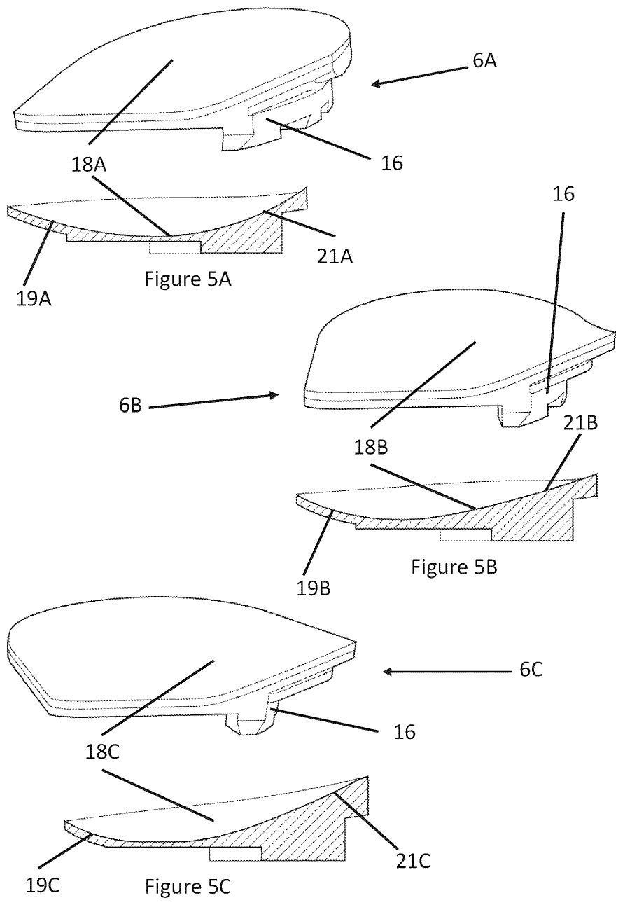

Referring to FIGS. 4B and 5A, a rear-foot component 6 is shown without the padded layer thereon. The component 6 may be integrally formed from a relatively rigid material capable of maintaining its shape beneath the weight of the wearer, such as such as PA11 Nylon or Polypropylene. The rear-foot component 6 comprises a base 16 having attachment features via which the rear-foot component 6 can be removably attached to the mid-foot component 4 (as will be discussed). An upper side of the rear-foot component defines a heel cup region 18 configured to support the wearer's heel. The heel cup region 18 comprises two heel-receiving surfaces 19, 21 extending generally laterally outwardly from a relatively flat centre 22 of the heel cup 18, one of which surfaces 21 may be angled (including more angled than the other surface 19), relative to a ground plane, to provide a corresponding level of heel correction to the wearer. In FIG. 4B, the surface 21 is angled from the horizontal to a relatively high extent (approximately 18 degrees) to provide a relatively severe degree of heel correction. Of course, other rear-foot components 6 of the kit 2 may have respective surfaces that are angled to other extents to provide more, less or no heel correction.

FIGS. 5A to 5C respectively show the three rear-foot components 6A-6C of FIG. 1, along with respective cross-sectional views taken along the line A-A of FIG. 4C. It should be noted that FIG. 4C shows the top view of each of the three rear-foot components 6A to 6C in FIG. 1. In other words, the three rear-foot components 6A-6C appear the same in plan view. FIG. 5A shows a first rear-foot component 6A does not provide any heel correction. For example, the heel cup region 18A may be substantially flat, or the angles of the laterally extending heel-receiving surfaces 19A, 21A of the heel cup region 18A relative to the ground are substantially identical. In other words, the shape of the heel cup region 18A is generally symmetrical about its longitudinal axis such that the rear-foot component 6A provides no heel correction.

FIG. 5B shows a second rear-foot component 6B comprising a heel cup region 18B having a heel-receiving surface that has a curvature that is asymmetrical. In particular, one of the heel-receiving surfaces 19B is angled to a small extent (similar to surface 19A of the first rear-foot component 6A), while the other heel-receiving surface 21B is angled to a first extent (e.g., approximately 8 degrees from the ground) to provide a mild level of heel correction.

FIG. 5C shows a third rear-foot component 6C with a heel cup region 18C having a first heel-receiving surface 19C that is angled to a small extent, while the corrective heel-receiving surface 21C is angled to a second extent (e.g., approximately 18 degrees from the ground) to provide a severe or high degree of heel correction. Each of these three rear-foot components 6A-6C can be removably attached to any one of the three mid-foot components 4A-4C, as shown in FIG. 2, to arrive at nine different orthotics 14, each with a unique combination of arch support and heel correction.

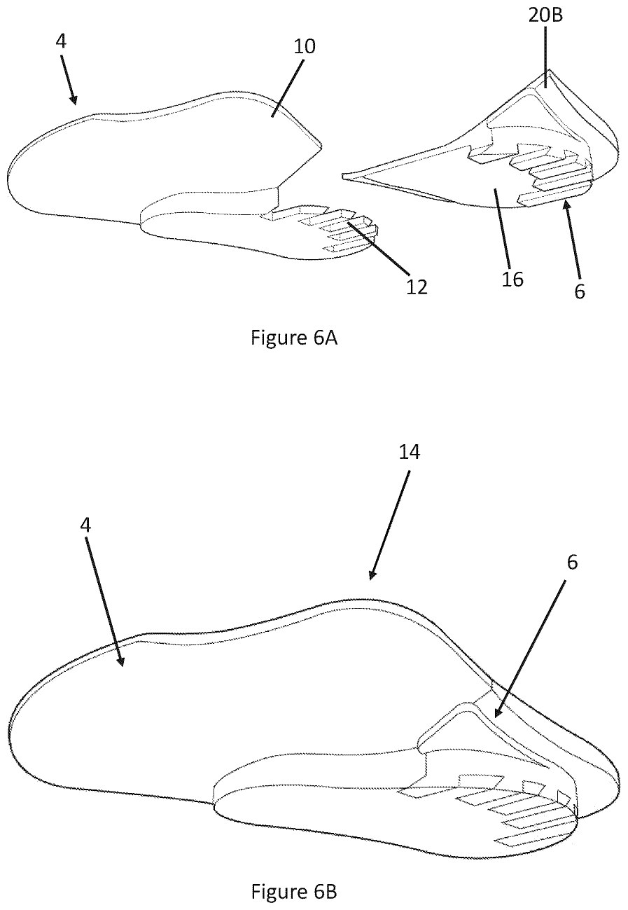

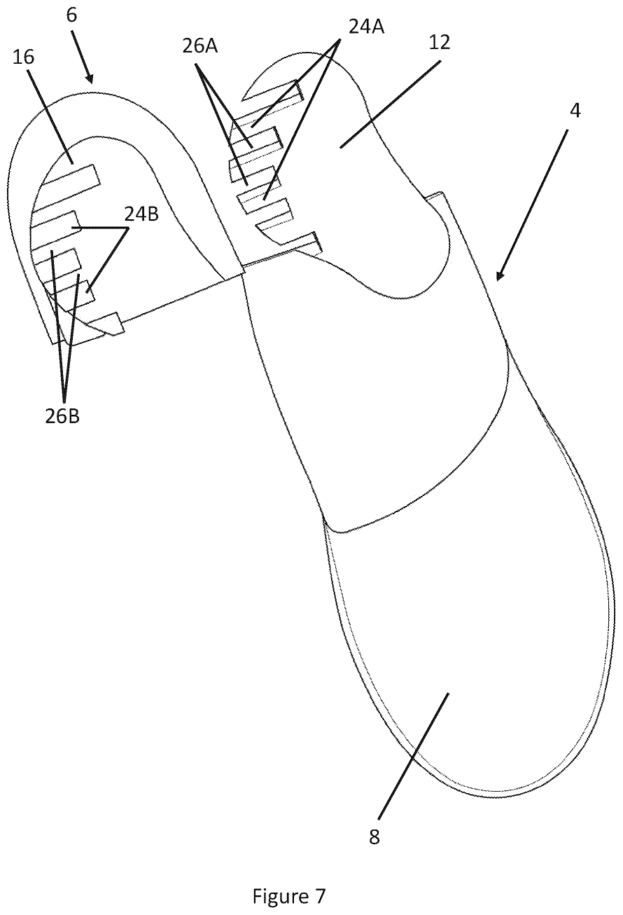

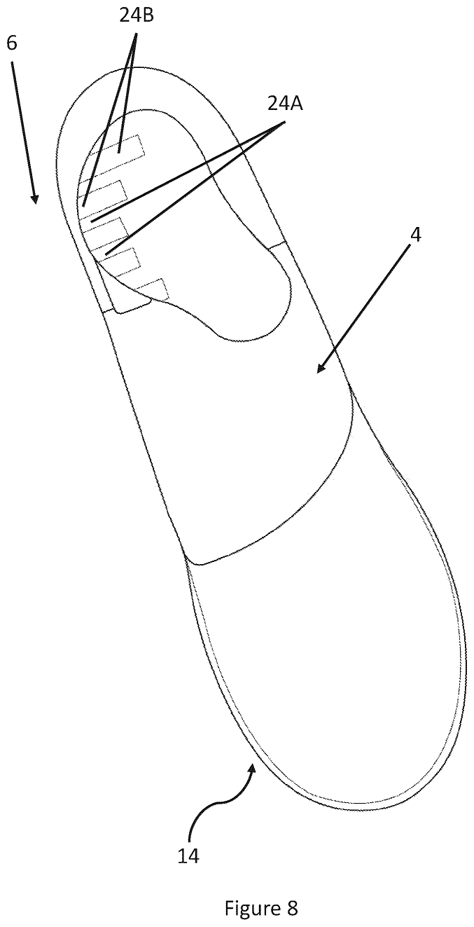

One way the mid-foot components 4 and the rear-foot components 6 can be removably attached will now be described with reference to FIGS. 6A to 8, though of course various other methods of connecting the components 4, 6 are considered within the scope of the present specification. As discussed, the mid-foot components 4 comprise a rearward region 12 having attachment features via which the rear-foot components 6 can be removably attached. In the depicted example, the rearward attachment features comprise a series of teeth 24A spaced apart by grooves 26A. The base 16 of the rear-foot component 6 also comprises a series of complementarily shaped and positioned teeth 24B spaced by grooves 26B. As such, a simple teeth-and-groove connection mechanism is provided wherein the teeth 24B of the rear-foot component 6 are insertable into respective grooves 26A of the mid-foot component 4, and the teeth 24A of the mid-foot component 4 are insertable into respective grooves 26B of the rear-foot component 6, thereby joining the two components 4, 6 so as to form the orthotic 14. In the present example, the teeth and grooves 24, 26 extend in a lateral direction that is generally perpendicular to the longitudinal axis of the orthotic 14. As such, the orthotic 14 is formed by bringing the mid and rear-foot components 4, 6 laterally toward one another such that their respective teeth 24 and grooves 26 mesh to form the orthotic 14.

Many modifications of the above embodiments will be apparent to those skilled in the art without departing from the scope of the present invention. For example, while certain and discrete levels of heel correction and arch support have been disclosed, the present kit may comprise any number of mid and rear-foot components providing respective levels of arch support and heel correction.

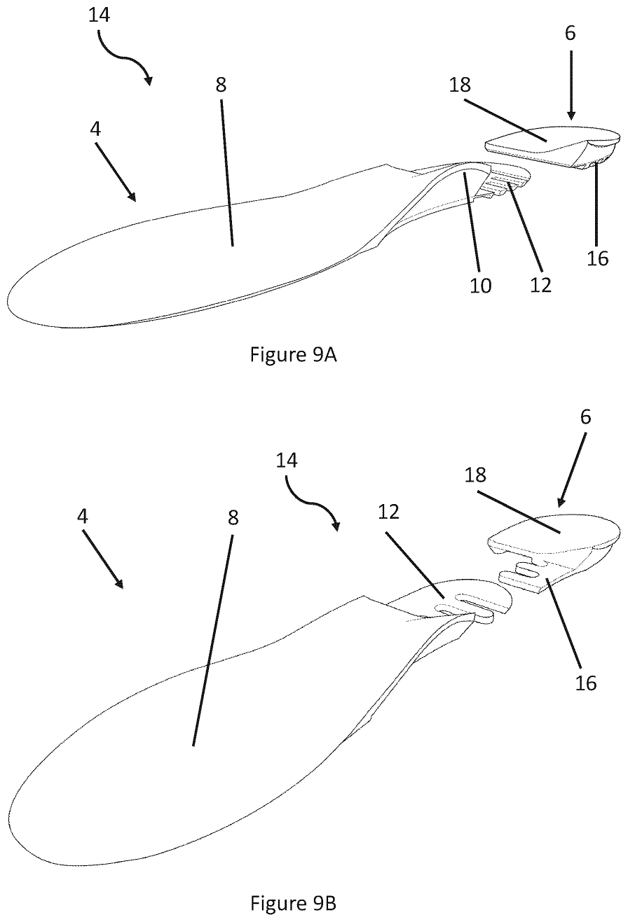

While certain materials have been disclosed, the present components may be configured and formed from any number of other suitable materials. For example, FIG. 9A shows an embodiment 14 in which the connective attachment features or portions 12, 16 of the respective mid and rear-foot components 4, 6 are formed from a harder material such as plastic, whereas the foot-receiving portions of the components (e.g., the landing 8 and middle support region 10 of the mid-foot component 4, and the heel cup 18 of the rear-foot component 6) are formed from a cushioning material such as foam. Meanwhile, FIG. 9B shows a version wherein both components 4, 6 are entirely made from a cushioning material, such as form.

Moreover, while the Figures show one manner of releasably connecting the mid and rear-foot components 4, 6, other forms of attachment are of course within the scope of the present specification, including tongue-and-groove connections and snap-fit interlocking mechanisms and the like.

It is also envisaged that one or more of the mid and/or rear-foot components 4, 6 can form the basis of a template device for assessing a patient's foot and the support/correction required. For example, several rear-foot components 4 providing increasing levels of heel correction may be arranged next to one another such that a patient can fit their heel into each component 6 to determine the level of heel correction that best suits their needs.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A kit for forming a foot orthotic, the kit comprising:
two or more mid-foot components, each having a support region that is arched to respective extents such that each mid-foot component provides respectively different levels of arch support; and
one or more rear-foot components configured to provide heel support,
wherein the or each rear-foot component is removably attachable to each of the mid-foot components to form respective orthotics providing differing levels of arch and/or heel support.

2. The kit of claim 1, comprising:
two or more rear-foot components, each having a heel cup region that is angled to respective extents such that each rear-foot component provides respectively different levels of heel correction.

3. The kit of claim 1, wherein each mid-foot component comprises a rearward region configured for removable attachment to the rear-foot component(s).

4. The kit of claim 1, wherein an underside of the mid-foot components and an underside of the rear-foot component(s) comprise respective complementarily shaped features to facilitate removable attachment between the mid and rear-foot components to form the respective orthotic.

5. The kit of claim 4, wherein the complementarily shaped features comprise teeth and corresponding grooves into which they are insertable.

6. The kit of claim 1, wherein the mid-foot components and the rear-foot component(s) are formed from a relatively rigid plastic material.

7. The kit of claim 1, wherein one or more of the mid-foot components comprise a relatively flat forward landing for receiving a front of the wearer's foot.

8. The kit of claim 1, wherein each component comprises an upper cushioning layer for receiving the wearer's foot.

9. The kit of claim 1, comprising:
a first mid-foot component, the support region of which is arched to a low extent so as to provide a low level of arch support;
a second mid-foot component, the support region of which is arched to a medium extent so as to provide a medium level of arch support; and
a third mid-foot component, the support region of which is arched to a high extent so as to provide a high level of arch support.

10. The kit of claim 9, comprising:
a first rear-foot component, the heel cup region of which is substantially flat so as to provide substantially no heel correction;
a second rear-foot component, the heel cup region of which is angled to a first extent so as to provide a mild level heel correction; and
a third rear-foot component, the heel cup region of which is angled to a second extent which is larger than the first extent so as to provide a high level of heel correction.

11. A foot orthotic formed from a kit according to claim 1.
